(19) United States
(12) Patent Application Publication
(10) Pub. No.: US 2020/0046653 A1
(43) Pub. Date: Feb. 13, 2020

(54) IRON CHELATOR THERAPY METHOD

(71) Applicant: THE BOARD OF TRUSTEES OF THE LELAND STANFORD JUNIOR UNIVERSITY, Stanford, CA (US)

(72) Inventor: Geoffrey C. Gurtner, Woodside, CA (US)

(21) Appl. No.: 16/533,547

(22) Filed: Aug. 6, 2019

Related U.S. Application Data
(60) Provisional application No. 62/715,712, filed on Aug. 7, 2018.

(51) Int. Cl. A61K 31/133; A61P 17/02; A61K 47/32; A61K 47/38; A61K 9/70

(52) U.S. Cl. CPC A61K 31/133; A61P 17/02; A61K 9/7084; A61K 47/38; A61K 47/32

(57) ABSTRACT

Compositions and methods are provided for the prevention and treatment of chronic wounds such as pressure ulcers by transdermal delivery of an agent that increases activity of HIF-1alpha in the wound. Wound healing ability reduced in aged subjects can be restored by use of the iron chelator formulations and methods to be similar to that of young subjects.

### CROSS REFERENCE To RELATED APPLICATION

This application claims benefit of U.S. Provisional Application No. 62/715,712, filed Aug. 7, 2018, which disclosure is incorporated herein by reference in its entirety.

### STATEMENT AS TO FEDERALLY SPONSORED RESEARCH

This invention was made with Government support under Grant No. R01-DK074095-13 awarded by the National Institutes of Health. The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

There are currently 46.2 million people in the United States over 65 years old. By 2060, this number is expected to rise to 98 million. Chronic wounds including venous leg ulcers, diabetic foot ulcers, arterial insufficiency and pressure ulcers disproportionately affect these elderly individuals and lead to substantial morbidity, mortality and expense for the healthcare system. While several therapies for wound healing exist, they are only moderately effective in preventing and treating impaired wound healing in aged patients. Thus, there is an urgent clinical need to understand wound healing in human aging so that effective therapies to treat wounds can be developed for this population segment.

The critical molecular and cellular pathways responsible for normal wound healing that are impaired by advanced age have been identified. Specifically, it has been suggested that destabilization of HIF-1alpha impairs neovascularization, local fibroblast function and distal progenitor cell recruitment, causing deficiencies in aged wound healing. To enhance hypoxia signaling during impaired wound healing, two FDA-approved drugs, dimethyloxalylglycine (DMOG) and deferoxamine (DFO), were tested by topical application in solution form on diabetic and aged mice with wounds. DFO solution was found to improve wound healing in both diabetic and aged mice. Furthermore, systemic delivery of DFO by intraperitoneal (IP) injections was found to improve survival of ischemic tissue in aged mice by enhancing new blood vessel formation. See, e.g., Chang, E. I. et al. Age decreases endothelial progenitor cell recruitment through decreases in hypoxia-inducible factor lalpha stabilization during ischemia (Circulation 116, 2818-2829, doi:10.1161/CIRCULATIONAHA.107.715847 (2007)); Fujiwara, T. et al. Age-associated intracellular superoxide dismutase deficiency potentiates dermal fibroblast dysfunction during wound healing (Exp Dermatol, doi:10.1111/exd.13404 (2017)); Fujiwara, T. et al. Extracellular superoxide dismutase deficiency impairs wound healing in advanced age by reducing neovascularization and fibroblast function (Exp Dermatol 25, 206-211, doi:10.1111/exd.12909 (2016)); Rennert, R. C. et al. Diabetes impairs the angiogenic potential of adipose-derived stem cells by selectively depleting cellular subpopulations (Stem Cell Res Ther 5, 79, doi:10.1186/scrt468 (2014)); Thangarajah, H. et al. HIF-1alpha dysfunction in diabetes (Cell Cycle 9, 75-79, doi:10.4161/cc.9.1.10371 (2010)); Thangarajah, H. et al. The molecular basis for impaired hypoxia-induced VEGF expression in diabetic tissues (Proc Natl Acad Sci USA 106, 13505-13510, doi:10.1073/pnas.0906670106 (2009)); Duscher, D. et al. Transdermal deferoxamine prevents pressure-induced diabetic ulcers (Proc Natl Acad Sci USA 112, 94-99, doi:10.1073/pnas.1413445112 (2015)); Duscher, D. et al. Fibroblast-Specific Deletion of Hypoxia Inducible Factor-1 Critically Impairs Murine Cutaneous Neovascularization and Wound Healing (Plast Reconstr Surg 136, 1004-1013, doi:10.1097/PRS.0000000000001699 (2015)); and Duscher, D. et al. Comparison of the Hydroxylase Inhibitor Dimethyloxalylglycine and the Iron Chelator Deferoxamine in Diabetic and Aged Wound Healing (Plast Reconstr Surg 139, 695e-706e, doi:10.1097/PRS.0000000000003072 (2017)).

While these earlier findings repeatedly demonstrated that DFO in solution improved wound healing, there was a need to develop a reliable and consistent delivery system that could be used clinically to deliver the drug through both intact and injured skin of patients with chronic wounds. A DFO transdermal drug delivery system (DFO-TDDS) was developed, which when applied topically, released the drug in a sustained manner through the impermeable stratum corneum into the dermis, as described in Duscher, D. et al. Transdermal deferoxamine prevents pressure-induced diabetic ulcers (Proc Natl Acad Sci USA 112, 94-99, doi:10.1073/pnas.1413445112 (2015)). DFO-TDDS was tested by application on ulcers in diabetic mice. DFO-TDDS both prevented and healed ulcers in diabetic mice, where abnormal glucose metabolism provides one stimulus to production of Reactive Oxidative Species (ROS), disrupting HIF-1alpha.

### SUMMARY OF THE DISCLOSURE

Chronic wounds are a significant medical and economic problem worldwide. Individuals over the age of 65 are particularly vulnerable to pressure ulcers and impaired wound healing. With this demographic growing rapidly there is a need for effective treatments. It had been shown that defective hypoxia signaling through destabilization of the master hypoxia-inducible factor 1a (HIF-1alpha) underlies impairments in both aging and diabetic wound healing. To stabilize HIF-1alpha, a transdermal delivery system of the FDA-approved small molecule deferoxamine (DFO) was developed, and it was found that transdermal DFO could both prevent and treat ulcers in diabetic mice. Transdermal delivery of an iron chelator such as DFO is shown to prevent pressure ulcers in aged animals and, importantly, normalize aged wound healing. Enhanced wound healing by the iron chelator, e.g., DFO, can be brought about by stabilization of HIF-1alpha and yield improvements in neovascularization. Suitable transdermal delivery vehicles for an iron chelator include gels, lotions, patches, etc., formulated for topical delivery. Transdermal iron chelator therapy with a molecule such as DFO can be rapidly translated into the clinic and may represent a new approach to prevent and treat pressure ulcers in aged patients.

In a first aspect, a method for restoring wound healing ability in a subject having a reduced wound healing ability due to age is provided, including: delivering an effective amount of an iron-chelating compound to the subject; increasing HIF-1alpha expression in the subject; and restoring the wound healing ability of the subject. Increasing the rate of wound healing may include increasing the rate of at least one of neovascularization and angiogenesis. In some variations, the rate of wound healing may be increased to be substantially equivalent to a rate of healing in a non-aged subject. In some variations, restoring the rate of wound healing may further include reducing formation of a pressure ulcer. In some variations, restoring the rate of wound healing may further include accelerating wound healing. In some variations, accelerating wound healing may include accelerating healing a pressure ulcer.

In some variations, the iron-chelating compound may be deferoxamine, deferiprone, or deferasirox. In some other variations, the iron-chelating compound may be deferoxamine.

Delivering the iron-chelating compound may include administering the iron-chelating compound transdermally. In some variations, administering the iron-chelating compound transdermally may include applying an extended release formulation of the iron-chelating compound to a susceptible region of a body part of the subject.

In some variations, the extended release formulation of the iron-chelating compound may include a biodegradable matrix. The biodegradable matrix may include a natural polymer, a synthetic polymer, or a combination of a natural polymer and a synthetic polymer.

In some variations, the extended release formulation of the iron-chelating compound may further include reverse micelles containing the iron-chelating compound. The reverse micelles may include a non-ionic surfactant. In some variations, the non-ionic surfactant may include one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In some variations, the non-ionic surfactant may include one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols; CTAB (cetyl trimethylammonium bromide); and sorbitan monolaurate (Span20). In some variations, the non-ionic surfactant may include at least one of Polysorbate 80 and sorbitan monolaurate (Span20). In some variations, the non-ionic surfactant may include at least one of Triglyceryl monooleate and cetyl alcohol. Each of the one or more non-ionic surfactants may be present in the extended release formulation at a concentration of 1% w/w to 25% w/w.

In some variations, the reverse micelles may further include polyvinylpyrrolidone. In some variations, polyvinylpyrrolidone may be present in the extended release formulation in 0.1 w/w % to 25 w/w %.

In some variations, the iron-chelating compound may be present within the extended release formulation in a concentration from 1% to 35% w/w. In another variation, the iron-chelating compound may be present with in the extended release formulation in a concentration of 13% w/w. In some variations, the iron-chelating compound may be delivered in any formulation described herein, and the formulation may incorporate any combination of components therein.

The extended release formulation of the iron-chelating compound may be disposed on an adhesive backing in variations of the method.

In another aspect, a formulation for transdermal delivery of an iron-chelating compound is provided, including an extended release formulation including a biodegradable polymer. In some variations, the iron-chelating compound may be deferoxamine, deferiprone, or deferasirox. In other variations, the iron-chelating compound may be deferoxamine. The biodegradable polymer may include a natural polymer, a synthetic polymer, or a combination of a natural polymer and a synthetic polymer. In some variations, the biodegradable polymer may include ethyl cellulose. In some variations, ethyl cellulose may be present at a concentration from 25%w/w to 75% w/w.

In some variations, the extended release formulation of the iron-chelating compound may further include reverse micelles including the iron-chelating compound.

The reverse micelles may include a non-ionic surfactant. In some variations, the non-ionic surfactant may include one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In some variations, the non-ionic surfactant may include TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols; CTAB (cetyl trimethylammonium bromide); and sorbitan monolaurate (Span20). In some variations, the non-ionic surfactant may include at least one of Polysorbate 80 and sorbitan monolaurate (Span20). In some variations, the non-ionic surfactant may include at least one of Triglyceryl monooleate and cetyl alcohol. In some variations, each of the one or more non-ionic surfactants may be present at a concentration of 1% w/w to 25% w/w.

In some variations, the reverse micelles may further include polyvinylpyrrolidone. In some variations, the polyvinylpyrrolidone may be present at a concentration of 0.1% to 25% w/w.

The iron-chelating compound may be present within the extended release formulation in a concentration from 1% to 35% w/w. In some variations, the iron-chelating compound may be present with in the extended release formulation in a concentration of 13% w/w.

In some variations, the extended release formulation of the iron-chelating compound may be disposed on an adhesive backing. In some variations, the extended release formulation is a lotion or gel.

In another aspect, a method of reducing formation of a pressure ulcer in a subject at risk thereof is provided, including: delivering an effective amount of an iron-chelating compound to the subject. In some variations, the iron-chelating compound may be deferoxamine, deferiprone, or deferasirox. In some variations, the iron-chelating compound may be deferoxamine.

In some variations, delivering the iron-chelating compound may include administering the iron-chelating compound transdermally. Administering the iron-chelating compound transdermally may include applying a extended release formulation of the iron-chelating compound to a susceptible region of a body part of the subject.

In some variations, the extended release formulation of the iron-chelating compound may include a biodegradable matrix. The biodegradable matrix may include a natural polymer, a synthetic polymer, or a combination of a natural polymer and a synthetic polymer.

In some variations, the extended release formulation of the iron-chelating compound may further include reverse micelles including the iron-chelating compound.

In some variations, the reverse micelles may include a non-ionic surfactant. The non-ionic surfactant may include one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In another variation, the non-ionic surfactant may include one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides);fatty alcohols; CTAB (cetyl trimethylammonium bromide); and sorbitan monolaurate (Span20). In some variations, the non-ionic surfactant may include at least one of Polysorbate 80 and sorbitan monolaurate (Span20). In some variations, the non-ionic surfactant may include at least one of Triglyceryl monooleate and cetyl alcohol. In some variations, each of the one or more non-ionic surfactants is present at a concentration of 1% w/w to 25% w/w.

In some variations, the reverse micelles may further include polyvinylpyrrolidone. In some variations, the polyvinylpyrrolidone may be present at a concentration of 0.1% to 25% w/w.

In some variations, the iron-chelating compound may be present within the extended release formulation in a concentration from 1% to 35% w/w. In some variations, the iron-chelating compound may be present with in the extended release formulation in a concentration of 13% w/w.

In some variations, the extended release formulation of the iron-chelating compound may be disposed on an adhesive backing.

In some variations, delivering an effective amount of an iron-chelating compound may further include modulating hypoxia inducible factor-1 alpha (HIG-1alpha).

In some variations, the subject at risk of developing a pressure ulcer may have a reduced level of hypoxia inducible factor-1 alpha (HIG-1alpha) expression compared to a healthy subject. In some variations, the reduced level of HIF-1alpha may be due to age. In some variations, the subject at risk of developing a pressure ulcer may be at risk of repeated incidences of ischemia and reperfusion of a body part.

In some variations, the iron-chelating compound may be delivered in any formulation described herein, and the formulation may incorporate any combination of components therein.

In another aspect, a method of accelerating wound healing in a subject is provided, including administering an effective amount of an iron-ch PVP)elating compound to the subject.

In some variations, the iron-chelating compound may be deferoxamine, deferiprone, or deferasirox. In some variations, the iron-chelating compound may be deferoxamine.

In some variations, delivering the iron-chelating compound may include administering the iron-chelating compound transdermally. Administering the iron-chelating compound transdermally may include applying a extended release formulation of the iron-chelating compound to a susceptible region of a body part of the subject.

In some variations, the extended release formulation of the iron-chelating compound may include a biodegradable matrix. The biodegradable matrix may include a natural polymer, a synthetic polymer, or a combination of a natural polymer and a synthetic polymer.

In some variations, the extended release formulation of the iron-chelating compound may further include reverse micelles including the iron-chelating compound.

In some variations, the reverse micelles may include a non-ionic surfactant. The non-ionic surfactant may include one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In another variation, the non-ionic surfactant may include one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides);fatty alcohols; CTAB (cetyl trimethylammonium bromide); and sorbitan monolaurate (Span20). In some variations, the non-ionic surfactant may include at least one of Polysorbate 80 and sorbitan monolaurate (Span20). In some variations, the non-ionic surfactant may include at least one of Triglyceryl monooleate and cetyl alcohol. In some variations, each of the one or more non-ionic surfactants is present at a concentration of 1% w/w to 25% w/w.

In some variations, the reverse micelles may further include polyvinylpyrrolidone. In some variations, the PVP may be present at a concentration of 0.1% to 25% w/w.

In some variations, the iron-chelating compound may be present within the extended release formulation in a concentration from 1% to 35% w/w. In some variations, the iron-chelating compound may be present with in the extended release formulation in a concentration of 13% w/w.

In some variations, the extended release formulation of the iron-chelating compound may be disposed on an adhesive backing.

In some variations, the subject at risk of developing a pressure ulcer may have a reduced level of hypoxia inducible factor-1 alpha (HIG-1alpha) expression compared to a healthy subject. In some variations, the reduced level of HIF-1alpha may be due to age. In some variations, the subject at risk of developing a pressure ulcer may be at risk of repeated incidences of ischemia and reperfusion of a body part.

In some variations, the iron-chelating compound may be delivered in any formulation described herein, and the formulation may incorporate any combination of components therein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the claims that follow. A better understanding of the features and advantages of the present invention will be obtained by reference to the following detailed description that sets forth illustrative embodiments, in which the principles of the disclosure are utilized, and the accompanying drawings of which:

FIG. 1A is a graphical representation of a pressure ulcer model in aged mice according to some embodiments of the disclosure.

FIG. 1B is a photographic representation of pressure ulcer wound prevention/healing in aged mice according to some embodiments of the disclosure.

FIG. 1C is a graphical representation of wound area of treated and untreated aged mice in pressure ulcer wound prevention/healing according to some embodiments of the disclosure.

FIGS. 1D to 1F are photographic and graphical representations of CD31 levels in untreated and treated aged mice according to some embodiments of the disclosure.

FIGS. 1G to 1I are photographic and graphical representations of apoptosis in treated and untreated aged mice according to some embodiments of the disclosure.

FIGS. 2A to 2H are photographic and graphical representations of excisional wound healing in treated aged mice compared to untreated aged mice and untreated young mice according to some embodiments of the disclosure.

FIGS. 3A to 3F are photographic and graphical representations of healed wounds and biochemical characteristics thereof in treated and untreated aged mice.

### DETAILED DESCRIPTION

As used herein, “treating” and “treatment” and the like, refer to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease, i.e., infection. The term “treatment” as used herein covers any treatment of a wound in a mammal, particularly a human, and includes: preventing a wound in an individual from dysfunction in initial healing; treating a wound that has reached a chronic state; or relieving chronic wound symptoms by mitigating or ameliorating the symptoms or conditions.

As used herein, “prophylaxis” refers to a measure or measures taken for the prevention or partial prevention of a disease or condition.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, “subject” includes mammals, e.g. cats, dogs, horses, pigs, cows, sheep, rodents, rabbits, squirrels, bears, primates such as chimpanzees, gorillas, and humans which may suffer from chronic wounds, particularly chronic skin ulcers. The term “subject” also comprises elderly individuals, diabetic individuals, etc., who may be at a higher risk for chronic wounds.

As used herein, “wound management” refers to therapeutic methods that induce and/or promote and/or accelerate repair of a wound, such as a pressure ulcer, including, but not limited to, arresting tissue damage such as necrotization, promoting tissue growth and repair, reduction or elimination of an established microbial infection of the wound and prevention of new or additional microbial infection or colonization. The term may further include reducing or eliminating the sensation of pain attributable to a wound. In some variations, wound management may further include reducing or substantially eliminating the potential for forming a pressure ulcer.

As used herein, “pharmaceutically acceptable” refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

As used herein, “carrier” refers to any pharmaceutically acceptable solvent of agents that will allow a therapeutic composition to be administered directly to a wound of the skin. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal.

Hypoxia-inducible factor (HIF-1) is an oxygen-dependent transcriptional activator, which plays crucial roles in the angiogenesis of tumors and mammalian development. The term “HIF-1”, as used herein, includes both the heterodimer complex and the subunits thereof, HIF-1alpha and HIF-1. The HIF 1 heterodimer consists of two helix-loop-helix proteins; these are termed HIF-1alpha, which is the oxygen-responsive component (see, e.g., Genbank accession no. Q16665), and HIF-1beta. The latter is also known as the aryl hydrocarbon receptor nuclear translocator (ARNT). In some variations, HIF-1alpha may refer to the human form of HIF-1alpha (see, e.g., Genbank Accession No. NM001530). The stability and activity of HIF-1alpha are regulated by various post-translational modifications, hydroxylation, acetylation, and phosphorylation. Under normoxia, the HIF-1alpha subunit is rapidly degraded via the von Hippel-Lindau tumor suppressor gene product (vHL)-mediated ubiquitin-proteasome pathway. The association of vHL and HIF-1alpha under normoxic conditions is triggered by the hydroxylation of prolines and the acetylation of lysine within a polypeptide segment known as the oxygen-dependent degradation (ODD) domain. During hypoxic conditions HIF-1alpha subunit becomes stable and interacts with coactivators such as p300/CBP to modulate its transcriptional activity.

HIF-1 acts as a master regulator of numerous hypoxia-inducible genes under hypoxic conditions. The heterodimer HIF-1 binds to the hypoxic response elements (HREs) of target gene regulatory sequences, resulting in the transcription of genes implicated in the control of cell proliferation/survival, glucose/iron metabolism and angiogenesis, as well as apoptosis and cellular stress. Some of these direct target genes include glucose transporters, the glycolytic enzymes, erythropoietin, and angiogenic factor vascular endothelial growth factor (VEGF).

Pressure ulcer prevention and treatment. Advanced age is a known risk factor for pressure ulcer formation. Elderly bed-ridden patients are at significant risk for the development of pressure ulcers and have a prolonged recovery period due to impairments in neovascularization. Recurrence rates are high, and in many cases, lead to amputations and mortality. Even when less severe, occurrence of pressure ulcers in this population leads to large impacts on the daily quality of life for the patient and present increasingly involved care and cost for the institution or family caring for the patient.

Repetitive cycles of ischemia and reperfusion characterize the pathophysiology of pressure ulcers, resulting in increased reactive oxygen species (ROS). These elevated levels of ROS induce oxidative tissue damage, partially through an iron-driven hydroxyl radical formation pathway. Various strategies for stabilizing and increasing HIG-1alpha have been employed to improve new blood vessel formation and wound healing, including HIG-1alpha gene transfection and delivery of stem and progenitor cells with increased HIG-1alpha activity. Iron-chelating molecules, which can stabilize HIG-1alpha and protect cells from free-radical injury, can be delivered through intact or broken skin. Once delivered through the intact or broken skin, the iron-chelating molecules can both reduce the likelihood of pressure ulcer formation and accelerate wound healing by restoring/enhancing wound healing ability in aged subjects. Applicants have discovered that transdermal delivery of an iron chelator can ameliorate healing defects in aged populations, by increasing neovascularization and VEGF expression, while also decreasing apoptosis. The wound healing ability (e.g., expression of HIF-1alpha, leading to increased neovascularization and angiogenesis) of the aged population may be restored to levels equivalent to wound healing ability of non-aged populations.

Methods. Methods for wound management/prevention are provided wherein a wound of a human or animal subject, e.g. a pressure skin ulcer, is contacted topically or transdermally with an effective amount of a therapeutic composition comprising an iron-chelating agent. In particular, the subject requiring wound management, including prevention, may be at risk of pressure ulcers due to a reduced ability to prevent or heal pressure ulcers, due to reduced wound healing ability. The reduced wound healing ability may be due to age, as is described herein.

The wound management may be preventive, that is, reducing the likelihood that a pressure ulcer may form, or the wound management may be treating an already formed pressure ulcer. Administration of the compositions of the present invention to a wound may provide accelerated wound repair with reduced sepsis. Even with chronic ulcers that have penetrated the dermal layer, reduction of pain sensation, more extensive and quicker tissue growth and less overall discomfort to the patient may result from the administration of the compositions described herein. Any body part likely to be affected may be treated in the methods, such as a heel, hip, ankle, buttock, shoulder blade, spine, tailbone, or back of an arm or a leg where contact is made with a support.

Pressure ulcers may be classed based on the depth of soft-tissue damage. Stage 1 ulcers manifest hyperemia, warmth, and induration. While an ulcer (a defect of skin into the dermis) is not present in Stage 1, ulceration will form if the course is not arrested and reversed. Stage 2 ulcers involve erosion (defect of epidermis) or true ulceration, but subcutaneous tissue is not exposed. Stage 3 and 4 ulcers have deeper involvement of underlying tissue with more extensive destruction. Prognosis is excellent for early-stage ulcers; neglected and late-stage ulcers pose risk of serious infection and nutritional stress and are difficult to heal. Patients do not always progress from lower to higher stages. Sometimes the first physical evidence of a pressure ulcer is a deep, necrotic Stage 3 or Stage 4 ulcer. When ulcers develop quickly, subcutaneous tissue can become necrotic before the epidermis erodes.

The timing of for administration of a therapeutic composition as described herein, e.g. a transdermal patch, a lotion, a gel or a film, may vary for prophylaxis or treatment. In general, it is desirable to apply a transdermal patch or dressing when a chronic wound is detected, e.g. reaches Stage 1 or Stage 2, although wounds presenting with more advanced stages will find benefit from the methods as well.

Additionally, a care team for a subject may determine that the subject is at risk of developing a pressure ulcer due to an acute/chronic illness or increased mobility deficit forcing bedrest or use of a wheelchair. A prophylactic application of the therapeutic composition may be initiated to increase the resistance of the subject's dermis to the likely insult of such prolonged pressures at susceptible tissues on body parts such as the heels, hips, ankles, buttocks, shoulder blades, spine, tailbone, or back of an arm or a leg where contact is made with a support. That is, prophylactic improvement of HIF-1alpha functionality, relative to the subject's age-related typically depressed levels of expression, can lead to a more robust response of neovascularization and/or angiogenesis to initiation of ischemic/reperfusion injury.

Before applying the therapeutic composition to the patient, when treating an existing pressure ulcer, rather than prophylaxis, the wound can be debrided to clean the wound of necrotic or infected tissue. Debridation may be mechanical by cutting or pulling away damaged tissue from the wound or, if readily inaccessible, other methods including, but not limited to, the application of sterile maggots may be used. Optionally, the wound may be prewashed before the application of the therapeutic composition using a composition comprising a buffering agent, detergent, etc.

The dosage of the iron chelator and the selection of formulation can determine the frequency of drug depletion in a transdermal patch or a film. For example, the transdermal patch or film can be applied and changed to a fresh patch/film every day, every other day, every third day, etc. When the therapeutic formulation is applied as a lotion or a gel dressing, the frequency of application may be about every 2 hr, about every 3 hr, about every 4 hr, about every 6 hr or more. Administration of the therapeutic composition may be continued from about 2 days to about 60 days, about 5 days to about 45 days, about 7 days to about 30 days or about 10 days to about 20 days. In some variations, the therapeutic compositions are administered for a selected period of days, which may be any of the periods of days described herein, followed by a selected period of days when no therapeutic composition is applied, and the administration cycle may be repeated one or more times.

The methods may improve the score of a skin ulcer by at least one stage, e.g. from a Stage 3 or a Stage 4, to a Stage 1 or Stage 2, and may provide an improvement leading to full healing of the wound. The time required for such healing is less than the time required for healing in the absence of the treatment methods, e.g. a wound may be healed in less than about 4 weeks, less than about 3 weeks, less than about 2 weeks, or less.

Iron-chelating molecules. Iron-chelating molecules suitable for use in the methods, formulations, and kits described herein typically include oxygen, nitrogen, or sulfur atoms that are capable of forming coordinate bonds with iron, which may be either Fe+2 or Fe+3. A number of iron-chelating molecules have been approved for use in treating iron overload associated with a wide variety of diseases. Three of the most commonly used iron chelators include deferoxamine (DFO, N′-[5-(Acetyl-hydroxy-amino)pentyl]-N-[5-[3-(5-aminopentyl-hydroxy-carbamoyl) propanoylamino[pentyl]-N-hydroxy-butane diamide), deferiprone (3-hydroxy-1,2-dimethylpyridin-4(1H)-one, or deferasirox (4-[(3Z,5E)-3,5-bis(6-oxo-1-cyclohexa-2,4-dienylidene)-1,2,4-triazolidin-1-yl] benzoic acid), each of which may be utilized in the methods and formulations described herein.

DFO is a small molecule FDA-approved iron-chelator for hemochromatosis in diseases such as thalassemia and sickle cell disease, and often provided as a mesylate salt. It has previously been shown that systemic delivery of DFO can improve new blood vessel formation by stabilizing HIF-1alpha and enhancing the survival of ischemic flaps in aged mice. It has also been shown that DFO solution dripped on wounds can enhance healing. However, pressure ulcers in humans begin with an intact epidermis, and to prevent them requires delivery through intact skin. It is very difficult to consistently deliver DFO, a hydrophilic small molecule, through the hydrophobic epidermis, which limits the efficacy of simple topical administration. Any proposed topical delivery of DFO such as an ointment or cream needs to overcome these biochemical constraints to be effective as a prophylactic therapeutic. The same transdermal formulation may be employed for wound healing where skin may be broken or intact. Therefore, a transdermal formulation (DFO-transdermal drug delivery system (TDDS)) has been developed, enclosing the DFO within nanoscale reverse micelles that can penetrate the hydrophobic epidermis and release the small molecule drug specifically into the dermis. In vitro characterization of DFO-TDDS using a Franz cell set-up showed uniformity of drug release into the human dermis.

Formulations. A formulation for transdermal delivery of an iron-chelating compound may be formulated as a patch, lotion, gel, or film. As mentioned above, the delivery may be across intact skin or may be through a wound such as a pressure sore, where one or more layers of the epidermis/dermis have been damaged.

Any of the iron chelator molecules, such as deferoxamine, deferiprone, or deferasirox may be formulated in a patch, thin film, gel or lotion. In some variations, the iron chelator may be deferoxamine and may be formulated as a patch. In some variations, the iron chelator may be deferoxamine and may be formulated as a thin film. In some variations, the iron chelator may be deferoxamine and may be formulated as a gel or lotion.

Iron Chelator molecule. In some variations, the iron chelator molecule, such as deferoxamine, deferiprone, or deferasirox, may be present at a concentration of at least about 1%, about 2%, about 3%, about 5% about 7.5%, and not more than about 35%; not more than about 20%; not more than about 15%, not more than about 12.5%w/w; or any number between the enumerated concentrations. In some variations, the iron chelator molecule may be present at a concentration of at about 13%, as weight/weight percent of polymer within the lotion, gel, film or patch.

The iron chelator molecule may be present within a film or patch at a concentration of about 0.1 to about 10 mg/cm², about 0.5 mg/cm² to about 10 mg/cm², about 0.5 mg/cm² to about 7 mg/cm², about 0.5 mg/cm² to about 5 mg/cm², about 1.0 mg/cm² to about 10 mg/cm², or about 1 mg/cm² to about 10 mg/cm².

The iron chelator molecule may be present within a gel or lotion at a concentration of about 0.1 mM, about 1 mM, about 10 nM, about 100 mM, about 500 mM, about 1000 mM, or any value therebetween.

The total dose of the iron chelator molecule, such as deferoxamine, deferiprone, or deferasirox, provided in a patch or film may be at least about 500 mg, at least about 1.0 g, and not more than about 6.0 g, not more than about 5.0 g, or not more than about 2.0 g, and may be from about 1.0 g to about 3.0 g, e.g. about 100 mg.

Reverse Micelles. The iron-chelating molecule formulations, including the extended release formulation of the iron-chelating compound, may include reverse micelles including the iron-chelating compound. Reverse micelles may be dispersed in the biodegradable polymer, such as ethyl cellulose or may be dispersed in a lotion or gel vehicle as described herein. Upon dissolution of the biodegradable polymer, the reverse micelles enter the stratum corneum and disintegrate. PVP dissolves and DFO is delivered to the dermis. Specifically, DFO migrates from the extended release transdermal delivery system to the skin following application. Once through the hydrophobic stratum corneum, the reverse micelles can then disintegrate in the more hydrophilic, aqueous environment of the dermis. Thus, a controlled release over a predictable time period may be achieved.

The reverse micelles may include a non-ionic surfactant. The nonionic surfactant can also provide for formation of reverse micelles, which advantageously aid in delivery of the iron chelator. Suitable surfactants for this purpose can include TWEEN 85® (Polyoxyethylene (20) Sorbitan Trioleate); phospholipids, e.g. lecithin; fatty acid esters such as Plurol® Oleique CC 497 (Gattefosse, Triglyceryl Monooleate); TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols such as cetyl alcohol; and CTAB (cetyl trimethylammonium bromide). Fatty acid esters are long chain aliphatic carboxylic acid, 4 carbons to 26 carbons in length, which are esterified with alcohols, which may include aliphatic alcohols or glycerols. Fatty alcohols are long chain primary alcohols generally having from about 4 carbons to about 26 carbons and are generally straight chain alcohols such as lauryl, strearyl, oleyl, and cetyl alcohols. In some variations, the reverse micelles may include at least one nonionic surfactant, which may be Polysorbate 80 and/or sorbitan monolaurate (Span20). In other variations, the reverse micelles may include at least one nonionic surfactant, which may be Triglyceryl monooleate. In yet other variations, the reverse micelles may include at least one of a fatty acid ester (e.g., Triglyceryl monooleate (Plurol® Oleique)) and a fatty alcohol (e.g., cetyl alcohol). The surfactant may be present at a concentration of from about 0.1 wt % to about 25 weight %, about 10 wt % to about 20 wt %, about 12 wt % to about 18 wt %, about 14 wt % to about 17 wt %, about 15 wt %, about 16 wt %, about 17 wt %, or any value between any of these specifically cited values. If more than one surfactant is included, such as two surfactants, the two surfactants may be present in about 1:10; about 1:8: about 1:6: about 1:4; about 1:2; or about 1:1 w/w ratio to each other.

Stabilizer. Owing to its hydrophilicity and tendency to crystallize, iron chelator compounds like DFO are especially well suited for delivery when complexed with Polyvinylpyrrolidone (PVP). PVP is known to stabilize drugs in an amorphous form and to promote permeation of hydrophilic molecules. PVP may also be included in the extended release formulation to stabilize the iron chelator molecule within the reverse micelles. For example, PVP may be present at a concentration of from about 0.1 w/w % to about 25 w/w %, about 7 w/w % to about 20 w/w %, about 8 w/w % to about 18 w/wt %, about 10 w/w % to about 16 w/w %, about 10 w/w %, about 12 w/w %, about 14 w/w %, about 16 w/w %.

Other molecules for prevention of crystallization. The formulations may alternatively or additionally comprise other molecules to prevent crystallization of the iron chelator within the formulation, which may prevent crystallization within a vehicle or within the reverse micelles within a vehicle such as a lotion, gel, film or patch. Such additives include, without limitation, one or more additives selected from octyldodecanol at a concentration of from about 1.5 to about 4% w/w of polymer; dextrin derivatives at a concentration of from about 2% to about 5% w/w of polymer; polyethylene glycol (PEG) at a concentration of from about 2% to about 5% w/w of polymer; polypropylene glycol (PPG) at a concentration of from about 2% to about 5% w/w of polymer; mannitol at a concentration of from about 2% to about 4% w/w of polymer; Poloxamer 407, 188, 401 and 402 at a concentration of from about 5% to about 10% w/w of polymer; and Poloxamines 904 and 908 at a concentration of from about 2% to about 6% w/w of polymer.

Biodegradable polymer and optional backing. Whether the formulation is a patch, thin film, gel or lotion, it may include an extended release formulation including a biodegradable polymer. The biodegradable polymer may include a natural polymer, a synthetic polymer, or a combination of a natural polymer and a synthetic polymer. Suitable biodegradable polymers useful in the formulation include hydrophilic gelling agents, including but not limited to, carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose (i.e., sodium carboxymethylcellulose 7H 4F) and polyethylene.

Medical dressings suitable for use in the methods of the present invention for contacting a wound with the therapeutic compositions can be any material that is biologically acceptable and suitable for placing over any chronic wound. In exemplary embodiments, the support may be a woven or non-woven fabric of synthetic or non-synthetic fibers, or any combination thereof. The dressing may also comprise a support, such as a polymer foam, a natural or man-made sponge, a gel as described above or a membrane that may absorb or have disposed thereon, the therapeutic formulation containing an iron chelator.

The biodegradable polymer may be present in the lotion, gel, film or patch in a concentration from about 25%w/w to about 75%w/w, about 35%w/w to about 65% w/w, about 40%w/w to about 60%w/w, about 45%w/w to about 55%w/w, about 45%w/w, about 48%w/w, about 50% w/w, about 51% w/w, about 52%w/w, about 53%w/w, about 54%w/w, or about 55%w/w. In some embodiments, the biodegradable polymer may be present in the film or path in a concentration from about 40%w/w to about 60%w/w, or about 50%w/w to about 55%w/w. In other embodiments, a lotion or gel may not include a biodegradable polymer, but may include any other suitable vehicle or components as described herein.

Plasticizer. A plasticizer may be, but is not required to be included in the formulations, particularly for a thin film or patch. Any suitable plasticizer may be used, including but not limited to chitosan, sorbitol, glycerol, polyethylene glycol 400, dibutyl sebacate, diethyl phthalate, vegetable oils, triacetin, acetylated monoglycerides. The plasticizer, if present, may be present in the formulation in a concentration of about 10%, about 20%, about 30% or about 40% w/w of the polymers. In some variations, di-n-Butyl phthalate may be used as a plasticizer at a concentration of about 30% weight-in-weight of polymers.

In some variations, the iron chelator molecule may be formulated in a therapeutic gel or lotion composition (e.g., formulation). The iron chelator formulation may include reverse micelles and the extended release components of the formulation as described herein, and may include any of the additional components as described herein such as stabilizers, permeation enhancers, crystallization inhibitors, and the like. The iron chelator lotion or gel formulation may include a therapeutically acceptable vehicle to act as a diluent, dispersant or carrier, so as to facilitate its distribution and uptake when the formulation is applied to the skin. Vehicles other than or in addition to water can include liquid or solid emollients, solvents, humectants, thickeners and powders.

For topical delivery, an iron chelator molecule, including but not limited to, deferoxamine embedded within a poloxamer gel (Pluronic® F127) provides an efficient and targeted means of delivery. Hydrogels responsive to external stimuli such as pH or temperature may be included. Hydrogels are based on different polysaccharides, such as alginate, cellulose, chitosan, and dextran, which in turn respond to different environmental stimuli. Specifically, a chitosan based hydrogel can be manipulated to respond to temperature and pH in wound healing applications. Likewise, poloxamers such as P188 can be employed as a drug delivery gel and has demonstrated cytoprotective effects in animal models.

The therapeutically acceptable vehicle may be present in 5% to 99.9%, preferably from 25% to 80% by weight of the composition, and can, in the absence of other adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the iron-chelating molecules are formulated in an emulsion, the proportion of the fatty phase may be from about 5% to about 80% by weight, and preferably from about 5% to about 50% by weight, relative to the total weight of the composition. Oils, emulsifiers and co-emulsifiers incorporated in the composition in emulsion form are selected from among those used conventionally in the cosmetic or dermatological field. The emulsifier and emulsifier may be present in the composition at a proportion from about 0.3% to about 30% by weight, or about 0.5% to about 20% by weight, relative to the total weight of the composition.

When the iron-chelating molecules are formulated as an oily solution or gel, the fatty phase may constitute more than about 90% of the total weight of the composition. Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Exemplary hydrocarbons which may serve as emollients are those having hydrocarbon chains anywhere from about 12 to about 30 carbon atoms. Specific examples include mineral oil, petroleum jelly, squalene and isoparaffins.

Extended Release Properties. In some variations, the extended release formulation may release the iron chelator compound over a period of about 4 hr, about 8 hr, about 12 hr, about 24 hr, about 4 8 hr or more. In some variations, the extended release formulation may be a controlled release formulation where the iron chelator is released in a predetermined pattern over a period of time, which may be about 2 hr, about 4 hr, about 8 hr, about 12 hr, about 24 hr, about 48 hr or more.

Preparation of the extended release film or film to be included in a patch. Conveniently, the reverse micelle structure can be generated by dissolving the film components, e.g. iron chelator molecule, PVP(a stabilizer for the chelator molecule), ethyl cellulose and surfactant(s) in a lower alcohol, e.g. ethanol, then drying on a hydrophobic surface to form a film, which can be adhered to a suitable backing for use in the methods of the disclosure. Alternatively, the reverse micelle structure may be obtained by dissolving the iron chelator molecule, PVP, ethylcellulose and surfactant(s) in chloroform and drying as mentioned. When preparing the extended release film or film material for a patch, the concentration of all of the components may be much lower in the solution/reaction mixture which is dried-down to form the film or patch. For example, the concentration of the biodegradable polymer, e.g., ethyl cellulose may be present in the reaction mixture at about 1% w/w to about 10% w/w, before evaporation and film formation, whereupon the concentration of the biodegradable polymer is as described above for the film or patch.

Other components. The formulation may further comprise additional agents involved in wound healing, e.g. transdermal penetration enhancers, anti-microbial agents, and the like.

Permeability enhancer. In some embodiments, the formulation may include a permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP), which may be provided at a weight/weight concentration of from about 0.1% to about 10%, from about 2.5% to about 7.5%, or about 5%.

Buffering agent. The therapeutic compositions of the present invention may additionally include a pharmaceutically acceptable pH buffering agent that preferably will maintain the pH of the composition, when delivered to the skin injury or skin lesion, to between about pH 7.0 and about pH 9.0. A pH buffering agent may be selected from, but is not limited to, Tris (hydroxymethyl) aminomethane (trometamol; TRIZMA base), or salts thereof, phosphates or any other buffering agent such as, for example, phosphate-buffered saline that is biologically acceptable. The buffering agent may have an effective dose of between about 5 mM and about 250 mM.

Antimicrobial. The compositions of the present invention may also comprise at least one antimicrobial agent. The infections that may be treated by the methods and compositions of the present invention may be any opportunistic infection of a wound by a bacterium, or a multiple infection of more than one species of bacteria. Microbial species that may cause infections include Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic E. coli, Enterotoxin-producing E. coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P. vulgaris, Pseudomonas aeruginosa, Rhodococcus equi, Salmonella enteridis, S. typhimurium, S. typhosa, Shigella sonnei, S. dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The action of the antimicrobial agent can be either bacteriostatic wherein the antibiotic arrests the proliferation of, but does not necessarily kill, the microorganism or the activity of the antibiotic can be bactericidal and kill the organism or a combination of activities. Antibiotics suitable for use in the wound management methods of the present invention include, but are not limited to, beta-lactams (penicillins and cephalosporins), vancomycins, bacitracins, macrolides (erythromycins), lincosamides (clindomycin), chloramphenicols, tetracyclines, aminoglycosides (gentamicins), amphotericins, cefazolins, clindamycins, mupirocins, sulfonamides and trimethoprim, rifampicins, metronidazoles, quinolones, novobiocins, polymixins, tetracyclines, and Gramicidins and the like and any salts or variants thereof.

Surfactant for cleaning or adjunct bactericides. The therapeutic compositions for use in the methods of wound management may also comprise a surfactant that can useful in cleaning a wound or contributing to bactericidal activity of the administered compositions. Suitable surfactants include, but are not limited to, phospholipids such as lecithin, including soy lecithin and detergents. Preferably, the surfactant selected for application to a wound or skin surface is mild and not lead to extensive irritation or promote further tissue damage to the patient.

Suitable nonionic surfactants which can be used are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkyloylamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. A preferred surfactant is polypropylene glycol ethoxylates with a preferred concentration of between about 5% wt % and about 25% wt %, for example Pluronic F-127 (Poloxamer 407). In other embodiments of the composition, the surfactant comprises lecithin with or without the addition of Pluronic F-127, the Pluronic F-127 being between about 2% and about 20 wt % for increasing the viscosity or gelling of the compositions.

Kits. Kits for treating a subject are provided, including a container including a therapeutic composition including an iron-chelating compound, which may be any iron-chelating compound described herein, and instructions for its use. The therapeutic composition may be formulated as any formulation described herein for delivering an iron-chelating compound, and may be a lotion, a gel, a thin film or a transdermal patch. The kit may include additional dressings or wound closures to be applied about the therapeutic composition. In some variations, the kit may include applicators for applying the therapeutic compositions to the affected region of the subject.

### EXPERIMENTAL

Materials: C57B1/6 mice were acquired from the National Institute on Aging (NIA, Bethesda, Md.). Young mice were 3 months of age, while aged mice were over 21 months of age. All mice were housed in the Stanford University Veterinary Service Center in accordance with NIH and institution-approved animal care guidelines. All procedures were approved by the Stanford Administrative Panel on Laboratory Animal Care.

Monolithic matrix-type TDDS containing DFO. All reagents used were analytic grade. DFO mesylate. Ethyl cellulose (3.5% by weight, Cat. # 200697, Sigma Aldrich, viscosity 22 cP) and PVP (Cat# PVP10, Sigma Aldrich, 0.5% by weight) was dissolved with 1% w/w DFO mesylate (Cat. # D9533-1G, Sigma Aldrich) in chloroform and the nonionic surfactants polysorbate 80 (Tween 80) and sorbitan monolaurate 20 (Span 20) (1% each, by weight) were added to form reverse micelles. Di-n-butylphthalate was used as a plasticizer (30% weight-in-weight of polymers). The solution was stirred vigorously until a fine suspension was achieved. This solution was then poured onto a sterile glass Petri dish and dried at room temperature. The uniform dispersion was cast onto a 2% polyvinyl alcohol backing membrane, dried at 40° C. for 6 hr, and cut with a 16-mm circular biopsy punch in equal-sized discs. Finally, the finished TDDS was attached to a contact adhesive (Tegaderm; 3M).

Statistical analysis. SPSS 12 software was used to perform univariate Student t-tests and multivariate ANOVA for wound healing analysis. Power analysis was accomplished using G-Power software (Parkville, Australia).

### EXAMPLE 1

### Prevention and Acceleration of Wound Healing in Pressure Ulcer Model

Similar to humans, aged mice demonstrate impaired wound regeneration, characterized by greater tissue necrosis and decreased blood vessel formation compared to youthful mice. FIG. lA shows a graphic of a pressure ulcer model used, where application of two ceramic magnets on the dorsum can induce a pressure ulcer, (See Duscher, D. et al. Transdermal deferoxamine prevents pressure-induced diabetic ulcers (Proc Natl Acad Sci USA 112, 94-99, doi:10.1073/pnas.1413445112 (2015), incorporated heren by reference in its entirety). Mice (n=4) at least 21 months of age were randomized into two groups where they were injected with either phosphate buffered saline (PB, control group) or DFO (treated group). DFO-TDDS could not be used in this experiment because it physically interfered with the application of the magnets which are essential to the pressure ulcer model. Intra-peritoneal Injections started the day before ulcer formation. An ischemia-reperfusion cycle was performed to create each pressure ulcer. Ischemia induction was stimulated with the application of two ceramic magnets for a period of six hours, followed by a six-hour reperfusion period initiated by removal of the magnets. Each mouse had two separate ulcers formed on their back. Injections with either phosphate buffered saline (PBS) or DFO were continued every other day in the respective groups until wound closure. All wounds were covered with an occlusive dressing (Tegaderm®, 3M).

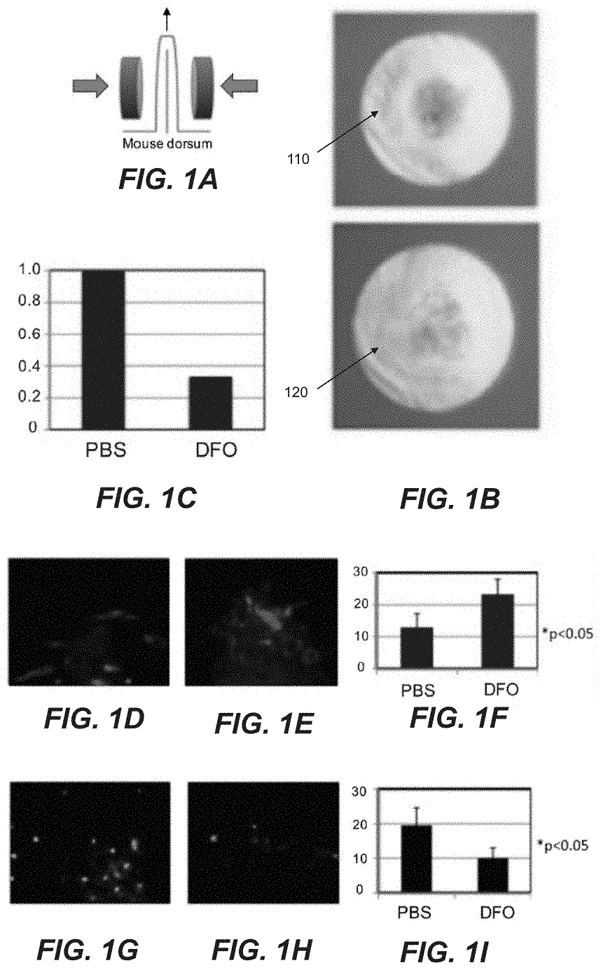

After the mice were euthanized, wounds were harvested with a 2-mm rim of unwounded skin. FIG.1B shows photographs of a representative wound 110 from the control group, injected only with PBS and a representative wound 120 from the DFO treated group. FIG. 1C shows the comparison in wound area between the control, PBS injected group, and the treated group, injected with DFO. It can be seen that the aged mice treated with systemic DFO demonstrated a decreased incidence of ulcer formation and lesser grade ulceration.

Frozen tissue samples for CD31 and terminal deoxynucleotidyl transferase dUTP Nick-End Labeling (TUNEL) immunohistochemistry were prepared by immediate OCT embedding (Sakura Finetek USA, Inc.). Trichrome staining was performed on sections of healed wounds that were either treated with DFO-TDDS or left untreated.

New blood vessel formation in the two groups was examined by performing immunohistochemistry for CD31 (Platelet endotheliean cell adhesion molecule) on histological sections of the pressure ulcers from the control and the treated group. The photograph in FIG. 1D shows CD31 immunofluorescence (red) for a representative section of healed wound for the control PBS-only group, and the photograph in FIG. 1E shows CD31 immunofluorescence (red) for a respective section of healed wound for the DFO-treated group. FIG. 1F is a graphical representation of the quantification of CD31 positive pixels per high power field (HPF). A statistically significant two-fold increase in neovascularization in the pressure ulcers from the mice treated with DFO (*p<0.05) was found. The histological sections of representative sections of healed wounds from both the control and the DFO-treated group were examined for apoptosis by TUNEL assay. The photograph in FIG. 1G shows immunofluorescence (green) for TUNEL for the control group and the photograph in FIG.1H shows immunofluorescence (green) for TUNEL for the DFO-treated group. FIG. 1I shows the quantification of TUNEL positive pixels per HPF for control (PBS only) and DFO-treated group, showing that treatment with DFO brought about a statistically significant two-fold decrease in apoptosis (*p<0.05). This suggested that DFO provided an efficient means of preventing and treating pressure ulcers in debilitated elderly patients.

### EXAMPLE 2

### Restoration of Wound Healing In Aged Mice

Transdermal DFO patches (DFO-TDDS) were formulated as above (See Duscher, D. et al. Transdermal deferoxamine prevents pressure-induced diabetic ulcers (Proc Natl Acad Sci USA 112, 94-99, doi:10.1073/pnas.1413445112 (2015), herein incorporated by reference in its entirety). Briefly, DFO is encapsulated within nonionic surfactants to generate reverse-micelles that allows for permeation through the stratum corneum. The reverse-micelles are dispersed throughout a degradable slow-release matrix which allows for continuous and efficient application of DFO into the dermis

An excisional wound model was used as described in Galiano, R. D., Michaels, J. T., Dobryansky, M., Levine, J. P. & Gurtner, G. C. Quantitative and reproducible murine model of excisional wound healing (Wound Repair Regen 12, 485-492, doi:10.1111/j.1067-1927.2004.12404.x (2004), herein incorporated by reference in its entirety). Briefly, 6-mm wounds were generated on the dorsa with the use of a biopsy punch. The wounds were stented open with silicone rings that were sutured to the mice with 6-0 sutures. Mice at least 21 months of age were randomized into two groups whereby one group received DFO-TDDS topically every day until closure and the other served as an untreated control. A third group of untreated young mice was also used as control.

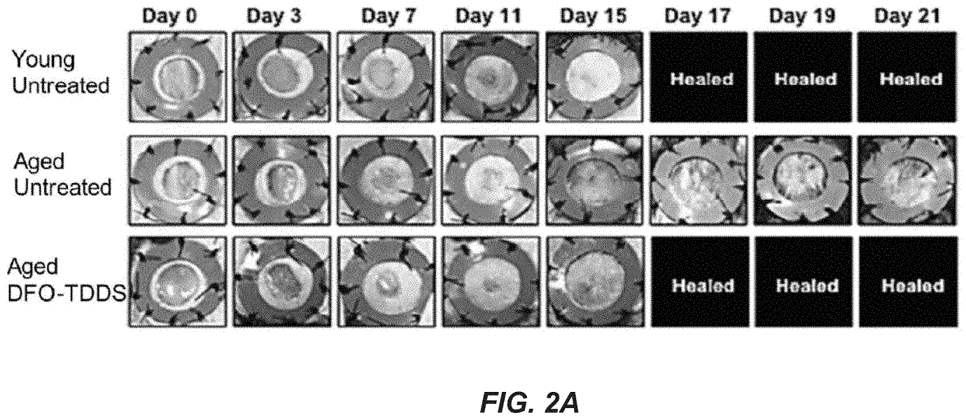

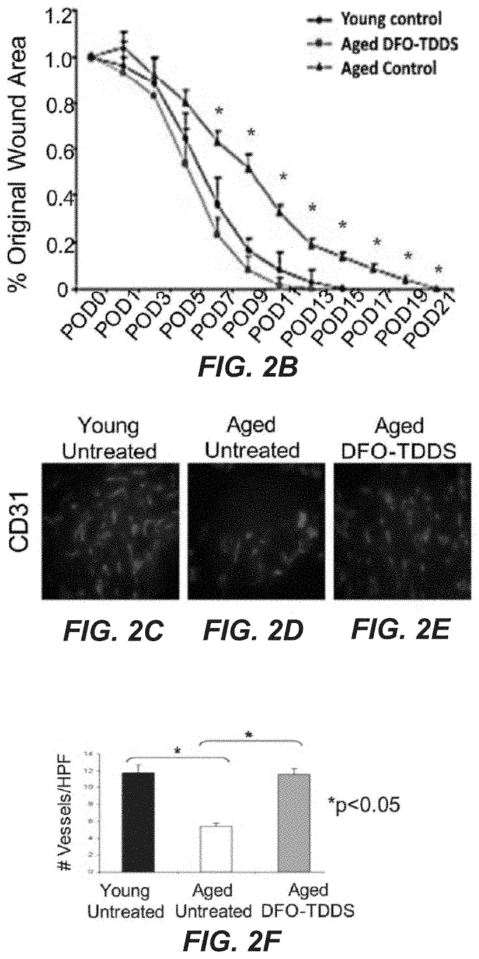

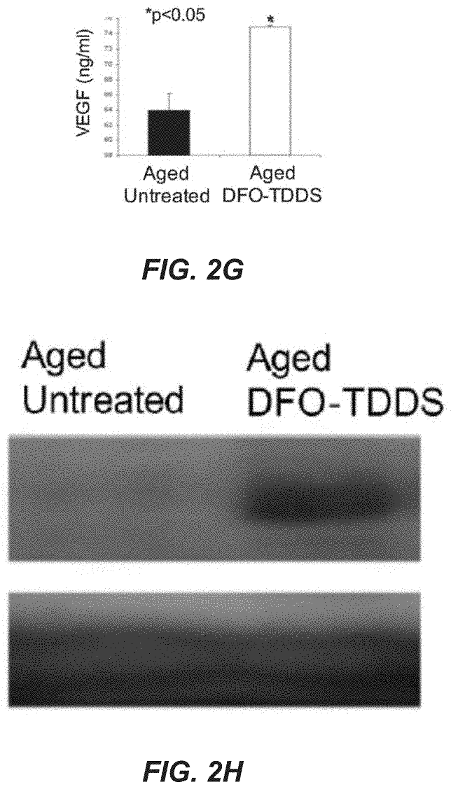

Wound closure of the DFO-TDDS treated group of aged mice was compared to wound closure in both the untreated young (3 months) and untreated aged (21 month) control groups (See FIG. 2A). Wounds in untreated aged mice closed significantly more slowly than those in the untreated young mice, consistent with previous findings. Twenty one days were required to see wound closure. Aged mice treated with DFO-TDDS showed significantly accelerated healing over the untreated aged control group, with closure in 15 days, which is equivalent to the closure period for untreated young mice. FIG. 2B shows wound healing curves as a function of time for these three groups, showing the DFO-TDDS treated group displaying similar time to wound closure as the untreated young mice control group. Where marked by “*”, the data points are statistically significant, e.g., p<0.05.

Following euthanasia of the mice at the completion of the wound closure period, skin samples were isolated from the healed wounds from each of the young untreated control group, the aged untreated control group, and the aged DFO-TDDS treated group, and used for molecular analysis.

Evaluation of CD31 immunohistochemistry immunofluorescence (red) displayed similar neovascularization between the young untreated mice (FIG. 2C) and the aged DFO-TDDS treated mice (FIG. 2E) while aged untreated mice (FIG. 2D) displayed significantly less staining. The number of vessels per high power field (HPF) in both of the young untreated and the aged DFO-TDDS treated group was found to be significantly higher (*p<0.05) than the aged untreated control following statistical analysis as shown in FIG. 2F.

Protein was isolated from harvested wounds from the aged untreated control group and the aged DFO-TDDS treated group via homogenization of the tissue in RIPA buffer combined with protease inhibitor. Vascular endothelial growth factor (VEGF) levels were measured with a VEGF ELISA (enzyme-linked immunosorbent assay) kit (R&D Systems, Minneapolis, MN), following the manufacturer's protocol. As shown in FIG. 2G, the DFO-TDDS treated group displayed significantly (*p<0.05) higher concentration of VEGF (over 74 ng/ml) compared to their untreated counterparts (about 64 ng/ml).

Western blot analysis was performed, taking protein on post-operative day 5 from the aged untreated control group and the aged DFO-TDDS treated group, was separated on a 4-12% polyacrylamide gel and then transferred to a nitrocellulose membrane. Anti-HIF-1alpha (ab179483 1:1000, Abcam, Cambridge, UK) and anti-β-actin (ab8227, Abcam, Cambridge, UK) were used as primary antibodies, while an HRP-conjugated secondary antibody was used at a 1:10,000 dilution (Abcam, Inc., Cambridge, UK) and detected using an ECL Plus Western Blotting Detection Kit (GE Healthcare, Chicago, Ill). As shown in FIG. 2H, DFO-TDDS treated mice show an increase (*p<0.05) in HIF-1alpha expression compared to their untreated aged counterparts.

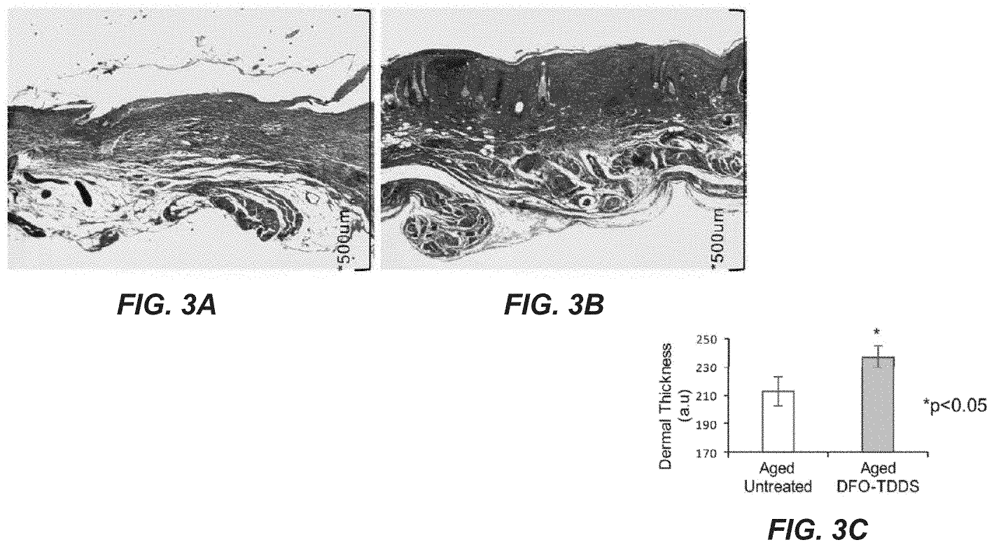

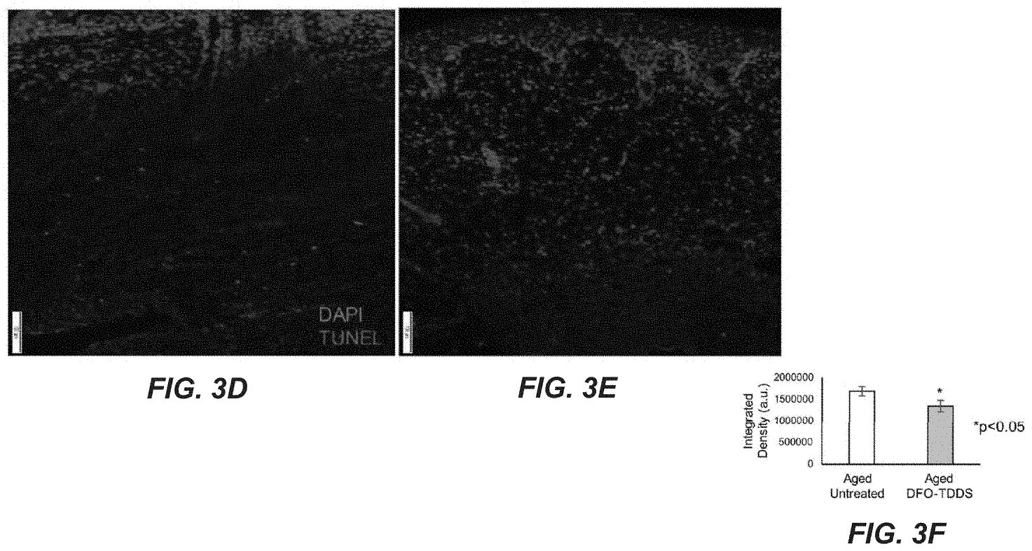

Histological sections of healed wounds in both aged untreated and aged DFO-TDDS groups were subjected to trichrome staining, as shown in FIG. 3A for aged untreated and FIG. 3B for aged DFO-TDDS treated, where the scale bar at the right side of each photograph is 500 um. Dermal thickness was calculated from the stained sections and represented in arbitrary units (a.u.) in FIG. 3C, and was over 230 a.u. for the DFO-TDDS treated group and over 210 a.u. for the untreated aged group. The dermal thickness was significantly higher in DFO-TDDS treated mice (*p<0.05) and DFO-TDDS treated wounds displayed re-appearance of epidermal appendages, indicating enhanced wound remodeling (See FIGS. 3A-B).Histological sections of healed wounds from aged untreated mice (FIG. D) and aged DFO-TDDS treated mice (FIG. 3E) were also subjected to TUNEL staining to label for DNA fragmentation and dying/dead cells (red) and counterstained with DAPI (blue). The fluorescence of TUNEL stain was quantified, and shown in FIG. 3F. (Scale bar: 10 um) There was significantly higher cell death (*p<0.05) in untreated wounds (over 150000 a.u. integrated density)compared to DFO-TDDS treated wounds (over 1000000 a.u.). Thus, we demonstrate that DFO-TDDS significantly accelerates wound healing in aged mice through increased HIF-1alpha expression and VEGF production. The healed wounds following DFO-TDDS treatment exhibit a significantly thicker dermis and decreased cell death. Treatment with DFO-TDDS enhances dermal thickness and wound remodeling in aged mice.

In summary, treatment with DFO in a transdermal delivery vehicle was shown to surprisingly restore wound healing ability to aged mice, providing wound healing ability equivalent to that of young mice. By increasing the level of HIF-1alpha, healing was accelerated to heal in the same period as young mice. Additionally, the healed wound had improved physiological and biochemcial characteristics, providing critical benefit for its potential in treatment of humans with similar debilitated healing abilities.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Section divisions in the specification are for ease of review only and do not limit any combination of elements discussed.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

List of some embodiments of the disclosure.

1. A method for restoring wound healing ability in a subject having a reduced wound healing ability due to age, including: delivering an effective amount of an iron-chelating compound to the subject; increasing HIF-1alpha expression in the subject; and restoring the wound healing ability of the subject.

2. The method of embodiment 1, wherein increasing the rate of wound healing includes increasing the rate of at least one of neovascularization and angiogenesis.

3. The method of embodiment 1 or 2, wherein the rate of wound healing is increased to be substantially equivalent to a rate of healing in a non-aged subject.

4. The method of any one of embodiments 1 to 3, wherein restoring the rate of wound healing further includes reducing formation of a pressure ulcer.

5. The method of any one of embodiments 1 to 4, wherein restoring the rate of wound healing further includes accelerating wound healing.

6. The method of embodiment 5, wherein accelerating wound healing includes accelerating healing a pressure ulcer.

7. The method of any one of embodiments 1-6, wherein the iron-chelating compound is deferoxamine, deferiprone, or deferasirox.

8. The method of any one of embodiments 1-7, wherein the iron-chelating compound is deferoxamine.

9. The method of any one of embodiments 1-8, wherein delivering the iron-chelating compound includes administering the iron-chelating compound transdermally

10. The method of embodiment 9, wherein administering the iron-chelating compound transdermally includes applying an extended release formulation of the iron-chelating compound to a susceptible region of a body part of the subject.

11. The method of embodiment 10, wherein the extended release formulation of the iron-chelating compound includes a biodegradable matrix.

12. The method of embodiment 11, wherein the biodegradable matrix includes a natural polymer, a synthetic polymer, or a combination of a natural polymer and a synthetic polymer.

13. The method of any one of embodiments 10-12, wherein the extended release formulation of the iron-chelating compound further includes reverse micelles including the iron-chelating compound.

14. The method of embodiment 13, wherein the reverse micelles include a non-ionic surfactant.

15. The method of embodiment 14, wherein the non-ionic surfactant includes one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In some variations, in the method of embodiment 14, the non-ionic surfactant includes one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); CTAB (cetyl trimethylammonium bromide); fatty alcohols; and sorbitan monolaurate (Span20).

16. The method of embodiment 14, wherein the non-ionic surfactants include at least one of Polysorbate 80 and sorbitan monolaurate (Span20). In some variations of the method of embodiment 14, wherein the non-ionic surfactant includes at least one of Triglyceryl monooleate and cetyl alcohol.

17. The method of any one of embodiments 14-16, wherein each of the one or more non-ionic surfactants is present in the extended release formulation at a concentration of 1% w/w to 25% w/w.

18. The method of any one of embodiments 13-17, wherein the reverse micelles further include polyvinylpyrrolidone.

19. The method of any one of embodiments 10-18, wherein the iron-chelating compound is present within the extended release formulation in a concentration from 1% to 35% w/w. In another variation, in the method of any one of embodiments 10-18, wherein the iron-chelating compound is present with in the extended release formulation in a concentration of 13% w/w.

20. The method of any one of embodiments 10-19, wherein the extended release formulation of the iron-chelating compound is disposed on an adhesive backing.

21. The method of any one of embodiments 1-20, wherein the iron-chelating compound is delivered in a formulation of any one of embodiments 22-38.

22. A formulation for transdermal delivery of an iron-chelating compound, including an extended release formulation including a biodegradable polymer.

23. The formulation of embodiment 22, wherein the iron-chelating compound is deferoxamine, deferiprone, or deferasirox.

24. The formulation of embodiment 22 or 23, wherein the iron-chelating compound is deferoxamine.

25. The formulation of any one of embodiments 22-24, wherein the biodegradable polymer includes a natural polymer, a synthetic polymer, or a combination of a natural polymer and a synthetic polymer.

26. The formulation of embodiment 25, wherein the biodegradable polymer includes ethyl cellulose.

27. The formulation of embodiment 26, wherein ethyl cellulose is present at a concentration from 25%w/w to 75% w/w.

28. The formulation of any one of embodiments 22-27, wherein the extended release formulation of the iron-chelating compound further includes reverse micelles including the iron-chelating compound.

29. The formulation of embodiment 28, wherein the reverse micelles include a non-ionic surfactant.

30. The formulation of embodiment 29, wherein the non-ionic surfactant includes one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In some variations, formulation of embodiment 29, the non-ionic surfactant includes TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); lecithin; fatty acid alcohols; CTAB (cetyl trimethylammonium bromide); and sorbitan monolaurate (Span20).

31. The formulation of embodiment 29 or 30, wherein the non-ionic surfactant includes at least one of Polysorbate 80 and sorbitan monolaurate (Span20). In some variations in the formulation of embodiment 29 or 30, wherein the non-ionic surfactant includes at least one of Triglyceryl monooleate and cetyl alcohol.

32. The formulation of any one of embodiments 29-31, wherein each of the non-ionic surfactants is present at a concentration of 1% w/w to 25% w/w.

33. The formulation of any one of embodiments 22-32, wherein the reverse micelles further include polyvinylpyrrolidone.

34. The formulation of embodiment 33, wherein the polyvinylpyrrolidone is present at a concentration of 0.1% to 25% w/w.

35. The formulation of any one of embodiments 22-34, wherein the iron-chelating compound is present within the extended release formulation in a concentration from 1% to 35% w/w. In another variation, in the formulation of any one of embodiments 22-34, wherein the iron-chelating compound is present with in the extended release formulation in a concentration of 13% w/w.

36. The formulation of embodiment 35, wherein the iron-chelating compound is present at a concentration of 13% w/w.

37. The formulation of any one of embodiments 22-36, wherein the extended release formulation of the iron-chelating compound is disposed on an adhesive backing.

38. The formulation of any one of embodiments 22-36, wherein the extended release formulation is a lotion or gel.

39. A method of reducing formation of a pressure ulcer in a subject at risk thereof, including: delivering an effective amount of an iron-chelating compound to the subject.

40. The method of embodiment 39, wherein the iron-chelating compound is deferoxamine, deferiprone, or deferasirox.

41. The method of embodiment 39 or 40, wherein the iron-chelating compound is deferoxamine.

42. The method of any one of embodiments 39-41, wherein delivering the iron-chelating compound includes administering the iron-chelating compound transdermally

43. The method of embodiment 42, wherein administering the iron-chelating compound transdermally includes applying a extended release formulation of the iron-chelating compound to a susceptible region of a body part of the subject.

44. The method of embodiment 43, wherein the extended release formulation of the iron-chelating compound includes a biodegradable matrix.

45. The method of embodiment 44, wherein the biodegradable matrix includes a natural polymer, a synthetic polymer, or a combination of a natural polymer and a synthetic polymer.

46. The method of any one of embodiments 43-45, wherein the extended release formulation of the iron-chelating compound further includes reverse micelles including the iron-chelating compound.

47. The method of embodiment 46, wherein the reverse micelles include a non-ionic surfactant.

48. The method of embodiment 47, wherein the non-ionic surfactant includes one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); and CTAB (cetyl trimethylammonium bromide). In another variation, in the method of embodiment 47, the non-ionic surfactant includes one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols, CTAB (cetyl trimethylammonium bromide); and sorbitan monolaurate (Span20).

49. The method of embodiment 47 or 48, wherein the non-ionic surfactant includes at least one of Polysorbate 80 and sorbitan monolaurate (Span20). In another variation, in the method of embodiment 47 or 48, wherein the non-ionic surfactant includes at least one of Triglyceryl monooleate and cetyl alcohol.

50. The method of any one of embodiments 47-49, wherein each of the non-ionic surfactants is present at a concentration of 1% w/w to 25% w/w.

51. The method of any one of embodiments 46-50, wherein the reverse micelles further include polyvinylpyrrolidone. In some variation, in the method of any one of embodiments 46-50, polyvinylpyrrolidone may be present in the extended release formulation in 0.1 w/w % to 25 w/w %.

52. The method of any one of embodiments 43-51, wherein the iron-chelating compound is present within the extended release formulation in a concentration from 1% to 35% w/w. In another variation, in the method of any one of embodiments 43-51, wherein the iron-chelating compound is present with in the extended release formulation in a concentration of 13% w/w.

53. The method of any one of embodiments 43-52, wherein the extended release formulation of the iron-chelating compound is disposed on an adhesive backing.

54. The method of any one of embodiments 39-53, wherein delivering an effective amount of an iron-chelating compound further includes modulating hypoxia inducible factor-1 alpha (HIG-1alpha).

55. The method of any one of embodiments 39-54, wherein the subject at risk of developing a pressure ulcer has a reduced level of hypoxia inducible factor-1 alpha (HIG-1alpha) expression compared to a healthy subject.

56. The method of embodiment 55, wherein the reduced level of HIF-1alpha is due to age.

57. The method of any one of embodiments 39-56, wherein the subject at risk of developing a pressure ulcer is at risk of repeated incidences of ischemia and reperfusion of a body part.

58. The method of any one of embodiments 39-57, wherein the iron-chelating compound is delivered in a formulation of any one of embodiments 22-38.

59. A method of accelerating wound healing in a subject, including administering an effective amount of an iron-chelating compound to the subject.

60. The method of embodiment 59, wherein the iron-chelating compound is deferoxamine, deferiprone, or deferasirox.

61. The method of embodiment 59 or 60, wherein the iron-chelating compound is deferoxamine.

62. The method of any one of embodiments 59-61, wherein delivering the iron-chelating compound includes administering the iron-chelating compound transdermally

63. The method of embodiment 62, wherein administering the iron-chelating compound transdermally includes applying a extended release formulation of the iron-chelating compound to a susceptible region of a body part of the subject.

64. The method of embodiment 63, wherein the extended release formulation of the iron-chelating compound includes a biodegradable matrix.

65. The method of embodiment 64, wherein the biodegradable matrix includes a natural polymer, a synthetic polymer, or a combination of a natural polymer and a synthetic polymer.

66. The method of embodiment 63 or 64, wherein the extended release formulation of the iron-chelating compound further includes reverse micelles including the iron-chelating compound.

67. The method of embodiment 66, wherein the reverse micelles include a non-ionic surfactant.

68. The method of embodiment 67, wherein the non-ionic surfactant includes one or more of TWEEN 85® (Polyoxyethylene (20) Sorbitan Trioleate); phospholipids; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p-,tert-octylphenoxyethoxyethanol); CTAB (cetyl trimethylammonium bromide)-TRPO (mixed trialkyl phosphine oxides); or CTAB (cetyl trimethylammonium bromide). In another variation, in the method of embodiment 67, the non-ionic surfactant includes one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty acid alcohols, CTAB (cetyl trimethylammonium bromide); and sorbitan monolaurate (Span20).

69. The method of embodiment 67 or 68, wherein the non-ionic surfactant includes at least one of Polysorbate 80 and sorbitan monolaurate (Span20). In another variation, in the method of embodiments 67 or 68, wherein the non-ionic surfactant includes t least one of Triglyceryl monooleate and cetyl alcohol.

70. The method of any one of embodiments 67-69, wherein each of the non-ionic surfactants is present at a concentration of 1% w/w to 25% w/w.

71. The method of any one of embodiments 66-70, wherein the reverse micelles further include polyvinylpyrrolidone. In some variation, in the method of any one of embodiments 66-70, polyvinylpyrrolidone may be present in the extended release formulation in 0.1 w/w % to 25 w/w %.

72. The method of any one of embodiments 63-71, wherein the iron-chelating compound is present within the extended release formulation in a concentration from 1% to 35% w/w. In another variation, in the method of any one of embodiments 63-71, wherein the iron-chelating compound is present within the extended release formulation in a concentration of 13% w/w.

73. The method of any one of embodiments 63-72, wherein the extended release formulation of the iron-chelating compound is disposed on an adhesive backing.

74. The method of any one of embodiments 59-73, wherein delivering an effective amount of an iron-chelating compound further includes modulating hypoxia inducible factor-1 alpha (HIG-1alpha).

75. The method of embodiment 74, wherein the subject has a reduced level of hypoxia inducible factor-1 alpha (HIG-1alpha) expression compared to a healthy subject.

76. The method of embodiment 75, wherein the reduced level of HIF-1alpha is due to age.

77. The method of any one of embodiments 59-76, wherein the iron-chelating compound is delivered in a formulation of any one of embodiments 22-38.

What is claimed is:

1. A method for restoring wound healing ability in a subject having a reduced wound healing ability due to age, comprising:
delivering an effective amount of an iron-chelating compound to the subject;
increasing HIF-1alpha expression in the subject; and
restoring the wound healing ability of the subject.

2. The method of claim 1, wherein increasing the rate of wound healing comprises increasing the rate of at least one of neovascularization and angiogenesis.

3. The method of claim 1, wherein the rate of wound healing is increased to be substantially equivalent to a rate of healing in a non-aged subject.

4. The method of claim 1, wherein restoring the rate of wound healing further comprises reducing formation of a pressure ulcer.

5. The method of claim 1, wherein restoring the rate of wound healing further comprises accelerating wound healing.

6. The method of claim 5, wherein accelerating wound healing comprises accelerating healing a pressure ulcer.

7. The method of claim 1, wherein the iron-chelating compound is deferoxamine, deferiprone, or deferasirox.

8. The method of claim 1, wherein the iron-chelating compound is deferoxamine.

9. The method of claim 1, wherein delivering the iron-chelating compound comprises administering the iron-chelating compound transdermally.

10. The method of claim 9, wherein administering the iron-chelating compound transdermally comprises applying an extended release formulation of the iron-chelating compound to a susceptible region of a body part of the subject.

11. The method of claim 10, wherein the extended release formulation of the iron-chelating compound comprises a biodegradable matrix.

12. The method of claim 11, wherein the biodegradable matrix comprises a natural polymer, a synthetic polymer, or a combination of a natural polymer and a synthetic polymer.

13. The method of claim 10, wherein the extended release formulation of the iron-chelating compound further comprises reverse micelles comprising the iron-chelating compound.

14. The method of claim 13, wherein the reverse micelles comprise a non-ionic surfactant.

15. The method of claim 14, wherein the non-ionic surfactant comprises one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols, and CTAB (cetyl trimethylammonium bromide).

16. The method of claim 14, wherein the non-ionic surfactant comprises at least one of Polysorbate 80 and sorbitan monolaurate (Span20).

17. The method of claim 14, wherein each of the one or more non-ionic surfactants is present in the extended release formulation at a concentration of 1% w/w to 25% w/w.

18. The method of claim 13, wherein the reverse micelles further comprise polyvinylpyrrolidone.

19. The method of claim 10, wherein the iron-chelating compound is present within the extended release formulation in a concentration from 1% to 35% w/w.

20. The method of claim 10, wherein the extended release formulation of the iron-chelating compound is disposed on an adhesive backing.

21. The method of claim 1, wherein the iron-chelating compound is delivered in a formulation of claim 22.

22. A formulation for transdermal delivery of an iron-chelating compound, comprising an extended release formulation comprising a biodegradable polymer.

23. The formulation of claim 22, wherein the iron-chelating compound is deferoxamine, deferiprone, or deferasirox.

24. The formulation of claim 22, wherein the iron-chelating compound is deferoxamine.

25. The formulation of claim 22, wherein the biodegradable polymer comprises a natural polymer, a synthetic polymer, or a combination of a natural polymer and a synthetic polymer.

26. The formulation of claim 25, wherein the biodegradable polymer comprises ethyl cellulose.

27. The formulation of claim 26, wherein ethyl cellulose is present at a concentration from 25%w/w to 75% w/w.

28. The formulation of claim 22, wherein the extended release formulation of the iron-chelating compound further comprises reverse micelles comprising the iron-chelating compound.

29. The formulation of claim 28, wherein the reverse micelles comprise a non-ionic surfactant.

30. The formulation of claim 29, wherein the non-ionic surfactant comprises one or more of TWEEN 85® (Polyoxyethylene (20) Sorbitan Trioleate); phospholipids; fatty acid esters;
TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols; and CTAB (cetyl trimethylammonium bromide).

31. The formulation of claim 29, wherein the non-ionic surfactant comprises at least one of Polysorbate 80 and sorbitan monolaurate (Span20).

32. The formulation of claim 30, wherein each of the one or more non-ionic surfactants is present at a concentration of 1% w/w to 25% w/w.

33. The formulation of claim 22, wherein the reverse micelles further comprise polyvinylpyrrolidone.

34. The formulation of claim 33, wherein the polyvinylpyrrolidone is present at a concentration of 0.1% to 25% w/w.

35. The formulation of claim 22, wherein the iron-chelating compound is present within the extended release formulation in a concentration from 1% to 35% w/w.

36. The formulation of claim 35, wherein the iron-chelating compound is present at a concentration of 13% w/w.

37. The formulation of claim 22, wherein the extended release formulation of the iron-chelating compound is disposed on an adhesive backing.

38. The formulation of claim 22, wherein the extended release formulation is a lotion or gel.
